(19) United States
(12) Patent Application Publication
(10) Pub. No.: US 2008/0070284 A1
(43) Pub. Date: Mar. 20, 2008

(54) Oxidative, Reductive, Hydrolytic and Other Enzymatic Systems for Oxidizing, Reducing, Coating, Coupling or Cross-Linking Natural and Artificial Fiber Materials, Plastic Materials or Other Natural or Artificial Monomer to Polymer Materials

(76) Inventor: Hans-Peter Call, Amsberg II (DE)

Correspondence Address: BACON & THOMAS, PLLC, 625 SLATERS LANE, FOURTH FLOOR, ALEXANDRIA, VA, 22314, US

(21) Appl. No.: 11/587,639

(22) PCT Filed: Apr. 26, 2005
(86) PCT No.: PCT/DE05/00762

§ 371 Date: Sep. 4, 2007

(30) Foreign Application Priority Data
Apr. 26, 2004 (DE) 10 2004 020 355.5

(51) Int. Cl. C12P 19/18

(52) U.S. Cl. 435/97

(57) ABSTRACT

The invention relates to methods for oxidizing (redox reactions, preferably pulp delignification/bleaching), for carrying out coupling reactions (grafting polymer materials) or for carrying out cross-linking reactions on natural (i.e. having natural origin) or artificial (i.e. synthetically produced polymers) monomers to polymers or of mixtures of natural and artificial polymers or of fibre materials, of lignocellulose-containing, cellulose-containing or protein-like natural polymers or fibre materials such as pulp, textiles like cotton and wool. The invention is characterized in that 1) these oxidation, coupling or cross-linking reactions are carrying out using hydrolases such as lipases, esterases, proteases, amidases, transferases, acylases, glycosidases or glycotransferases or oxidoreductases, such as preferably peroxidases, chloroperoxidases and laccases, either individually or in combination with one another; and 2) that these reactions ( oxidation, coupling or cross-linking reactions) are carrying out with the above mentioned substances and/or with property-changing substances such as monomer to polymer substances (natural or synthetic) either simultaneously or one after the other using specific enzyme-activated enhancer substances and/or coupling substances.

### STATE-OF-THE-ART

It is known, that hydrolytic enzymes like glycosidases and glycotransferases, other transferases (e.g. transglutaminases), lipases, esterases, proteases , amidases, acylases and oxidoreductases, such as preferably laccases and peroxidases may be able to couple enzymatically special enzyme substrates via generating of ester or ether bounds or via radical reactions.

Lipases and other esterases are particularly used for enantioselective catalysis and here ester hydrolysis as well as ester formation are of interest.

Other enzymes such as laccases, peroxidases are used as radical chain initiators e.g. for the production of wood-containing polymer materials like different board types (fibre boards, particle boards etc.).

Until now no pure enzymatic system has been described which is able to perform also in a water containing environment in a sufficient quantity coupling reactions (grafting of polymer materials) and/or cross-linking reactions on natural, (i.e. having natural origin) or artificial (i.e. synthetically produced) monomers to polymers or of mixtures of natural and artificial polymers or fibre materials. Fibre material means preferably lignocellulose-containing, cellulose-containing or protein-like natural polymers or fibre materials such as pulp or textiles like cotton and wool.

The normally performed chemical reactions (coupling reactions, cross-link reactions, blocking reactions of specific relevant groups etc.) work with the aid of coupling substances such as e.g. aldehydes, anhydrides, hydrazides, acryl derivatives, vinyl derivatives, oxiran-containing compounds, N-Hydroxysuccimidyl-compounds, halide-containing compounds like chlortriazines and other substances. These coupling compounds useful for cross-linking reactions bear at least two or more active coupling groups being able to react with relevant functional groups of the substances which have to be coupled like particularly amines, thiol groups, hydroxyl- or carboxyl groups.

Due to their high reactivity these compounds show often a high toxicity. Other disadvantages are the (in many cases) high costs due to high charges and long reaction times.

### GENERAL DESCRIPTION OF THE INVENTION

In order to overcome the mentioned difficulties and drawbacks of the state-of-the-art methods enzyme-based systems are provided which mainly distinguish by their significantly higher specificy, their faster reactions and lower toxicity.

Therefore they allow operations at lower costs and environmental safety.

It could be surprisingly found that using (in a specific purposeful manner) thise enzyme-based methods containing lipases, esterases, proteases, amidases, transferases, acylases, glycosidases, glycotransferases and oxidoreductases as preferably peroxidases, chloroperoxidases and laccases, either individually or in combination with special (redox) enhancer compounds according to the invention oxidizing reactions (redox reactions) can be performed as for example bleaching reactions including delignification of pulps.

It could also be surprisingly found that with the aid of these mentioned enzyme-based methods coupling and/or cross-linking reactions can be carried out with special coupling and/cross-linking enhancer compounds which can be activated by the enzymes used. Preferably the compounds which can be modified by such coupling reactions are mainly natural, (i.e. having natural origin) or artificial (i.e. synthetically produced) monomers to polymers or mixtures of natural and artificial polymers or fibre materials. Fibre material means preferably lignocellulose-containing, cellulose-containing or protein-like natural polymers or fibre materials such as pulp or textiles like cotton and wool.

Preferably the compounds which are coupled and/or cross-linked with the compounds which should be modified are the same or similar substances such as natural, (i.e. having natural origin) or artificial (i.e. synthetically produced) monomers to polymers or mixtures of natural and artificial polymers or fibre materials (preferably lignocellulose-containing, cellulose-containing or protein-like natural polymers) or of substances belonging to other below mentioned substance groups like e.g. UV absorbing substances, radical scavengers, etc.

In this respect an important application is the yellowing inhibition of pulps caused by the influence of light, and/or oxygen and/or temperature. This is particularly important for pulps such as high yield pulps (TMP, CTMP, BCTMP or groundwood etc.) which contain high amounts of lignin the main reason for the yellowing problem.

With the aid of the enzyme-based systems according to the invention UV absorbing substances (so-called “sunscreens”) such as e.g. benzotriazole compounds, p-aminobenzoic acids and derivatives, cinnamic acid and derivatives, 2-phenylbenzimidazole compounds and derivatives, dibenzoylmethane compounds and derivatives and benzophenone compounds like 2-Hydroxy-4-methoxy-benzophenone and derivatives and other relevant compounds are coupled onto the pulp fibres preferably through OH-groups of the cellulose and/or hemicellulose part of the fibres. Other UV-absorbing compounds according to the invention are described in: Klessinger and Michl: Lichtabsorption u. Photochemie organischer Moleküle, VCH, 1989.

Other important and relevant substances which can be coupled with the aid of the enzyme-based systems according to the invention for yellowing inhibition onto the fibres are radical scavengers such as nitroxyl radicals like TEMPO-compounds, nitrones, other NO-compounds or generally suitable antioxidants ( sulfide compounds, disulfide compounds, thiol compounds, ascorbic acid derived compounds etc.).

The preferred coupling group within the pulp fibres are the mentioned OH-radicals of the cellulose/hemicellulose part.

An other important application is the so-called “fibre modification” of pulps (but also e.g. textile materials).

With the aid of the enzyme-based systems according to the invention the compounds which are coupled and/or cross-linked with the compounds which should be modified, i.e. compounds which are coupled onto e.g. the fibres of pulps or textile fibres etc. are e.g. property-changing substances such as special monomer to polymer substances like hemicellulose (e.g xylans, other polysaccharides but also proteins or lignin compounds etc.). This treatment can cause a strong enhancement of the fibre strength.

These mentioned enzymatically catalysed reactions according to the invention are usable for general coupling, for general cross-linking and for blocking reactions of unwanted reactive groups or for combined reactions.

The present invention provides enzyme-based methods

A) for oxidizing reactions (redox reactions) preferably of pulps (delignification and/or bleaching) and

B) for coupling and/or cross-link reactions, characterized in, that

a) these oxidation, coupling or cross-linking reactions are carrying out using hydrolases such as lipases, esterases, proteases, amidases, transferases, acylases, glycosidases, glycotransferases or using oxidoreductases, such as preferably peroxidases, chloroperoxidases and laccases, either individually or in combination with one another, and characterized in, that

b) the oxidizing reactions are performed using the mentioned enzymes together with special (redox) enhancer compounds and characterized in that

c) these coupling and /or cross-linking reactions are carrying out with compounds which should be modified such as natural (i.e. having natural origin) or artificial (i.e. synthetically produced) monomers to polymers or mixtures of natural and artificial polymers or fibre materials (preferably lignocellulose-containing, cellulose-containing or protein-like natural polymers)

in combination with compounds which are coupled and/or cross-linked such as the same or similar compounds like natural, (i.e. having natural origin) or artificial (i.e. synthetically produced) monomers to polymers or mixtures of natural and artificial polymers or fibre materials (preferably lignocellulose-containing, cellulose-containing or protein-like natural polymers),

especially with property-changing compounds such as monomer to polymer substances (natural or synthetic) either simultaneously or one after the other and/or with compounds belonging to other substance groups like e.g. UV absorbing substances, radical scavengers, etc., and characterized in, that

d) special coupling and/or cross-linking enhancer compounds or coupling and/or cross-linking precursor compounds which are the coupling and/or cross-linking agents activated by the enzymes are used.

### DETAILED DESCRIPTION OF THE INVENTION

As enzymatic component used in the enzyme based systems accordingly the invention enzymes according to the International Enzyme-Nomenclature: Committee of the International Union of Biochemistry and Molecular Biology (Enzyme Nomenclature, Academic Press, Inc., 1992, pp. 306-337) are used, preferably enzymes of the class 3 (Hydrolasen) 3.1, 3.1.1, 3.1.2, 3.1.3, 3.1.4 and 3.1.7 such as e.g.:

carboxyl ester hydrolases (3.1.1), thiol ester hydrolases (3.1.2), phosphoric acid monoester hydrolases (Phosphatases) (3.1.3), phosphoric acid diester hydrolases (3.1.4), diphosphoric acid monoester hydrolases (3.1.7)

Particularly preferred among these are enzymes of group 3.1.1.3 lipases (triacylglycerol lipases, triglycerolacyl hydrolases).

Further enzymes, which split carbon/nitrogen (C/N) bonds (other than peptide bonds) are also preferred (3.5), particularly preferred are enzymes of group 3.5.5.1 nitrilases, of group 3.5.1.4 amidases and 3.5.1.14 acylases.

Also particularly preferred are enzymes of group 3.4 which act hydrolytically on peptid linkages, particularly 3.4 11-19 which belong to the exopeptidases and particularly preferred also the group 3.4. 21-24 and 3.4. 99, which contain the endopeptidases and here preferably the serine proteinases such as:

chymotrypsin (3.4.21.1), trypsin (3.4.21.4), subtilisin (3.4.21.62) and endopeptidase K (3.4.21.64);

also prefeered are enzymes of the group: cystein endopeptidases such as: papain (3.4.22.2), ficain (ficin) (3.4.22.3); bromelaine (3.4.22.32/3.4.22.33), and also preferred enzymes of the group: aspartic endopeptidases as:

pepsin (3.4.23.1/3.4.23.2); renin (3.4.23.15), aspergillopepsin (3.4.23.18/3.4.23.19), penicillopepsin (3.4.23.20); rhizopuspepsin (3.4.23.21); endothiapepsin (3.4.23.22); mucorpepsin (3.4.23.23); candidapepsin (3.4.23.24), saccharopepsin (3.4.23.25); rhodutorulapepsin (3.4.23.26); physaropepsin (3.4.23.26); acrocylindropepsin (3.4.23.28), polyporopepsin (3.4.23.29); pycnoporopepsin (3.4.23.30); scytalidopepsin A/B (3.4.23.31/3.4.23.32), xanthomonaspepsin (3.4.23.33);

and also peferred the group: metallo-endopeptidases such as:

microbial collagenase (3.4.24.3); gelatinase A/B (3.4.24.24/3.4.24.35); thermolysin (3.4.24.27); bacillolysin (3.4.24.28); deuterolysin (3.4.24.39);

Particularly preferred are also enzymes of the class 1 (oxidoreductases) according to the International Enzyme Nomenclature: Committee of the International Union of Biochemistry and Molecular Biology (Enzyme Nomenclature, Academic Press, Inc., 1992, pp. 24-154) among which the following are particularly preferred:

cellobiose: quinone-1-oxidoreductase 1.1.5.1, bilirubin oxidase 1.3.3.5, cytochrome oxidase 1.9.3, oxygenases, lipoxygenases, cytochrome P450 enzymes 1.13 and 1.14, superoxide dismutase 1.15.11, ferrioxidase, for example, ceruloplasmin 1.16.3.1;

especially preferred being the enzymes of group 1.10 which act on biphenols and related compounds. They catalyze the oxidation of biphenols and ascorbates. Suitable acceptors are NAD⁺, NADP⁺ (1.10.1), cytochrome (1.10.2), oxygen (1.10.3) or others (1.10.99).

Among these, particularly preferred are the enzymes of group 1.10.3 with oxygen (O₂) as acceptor.

Particularly preferred among the enzymes of this group are catechol oxidase (tyrosinase) (1.10.3.1), L-ascorbate oxidase (1.10.3.3), O-aminophenol oxidase (1.10.3.4) and laccase (benzenediol:oxygen oxidoreductase) (1.10.3.2), the laccases (benzenediol:oxygen oxidoreductase) (1.10.3.2) being particularly preferred.

Other particularly preferred enzymes are those of group 1.11 which act on a peroxide as acceptor. Especially preferred here are cytochrome C peroxidases (1.11.1.5), catalase (1.11.1.6), peroxidase (1.11.1.7), iodide peroxidase, (1.11.1.8), glutathione peroxidase (1.11.1.9), chloride peroxidase (1.11.1.10), L-ascorbate peroxidase (1.11.1.11), phospholipid hydroperoxide glutathione peroxidase (1.11.1.12), manganese peroxidase (1.11.1.13) and diarylpropane peroxidase (ligninase, lignin peroxidase) (1.11.,1.14).

Particularly preferred are peroxidases (1.11.1.7), chloroperoxidases (1.11.1.10) and catalases (1.11.1.6).

Additionally preferred are glycotransferases and transglutaminases of the class 2, group 2.3 und 2.4 und glycosidases of the class 3, group 3.2, also according to the International Enzyme-Nomenclature: Committee of the International Union of Biochemistry and Molecular Biology (Enzyme Nomenclature, Academic Press, Inc., 1992, pp. 24 to 154).

Peroxides necessary as co-substrates for some enzymes are added directly as H₂O₂, as organic peroxide compounds, as peroxide adducts (e.g. urea) or enzymatically generated.

The preferred enzymes for the generation of peroxide are oxidases with O₂ as acceptor of the class 1, group 1.1.3 according to the International Enzyme-Nomenclature: Committee of the International Union of Biochemistry and Molecular Biology (Enzyme Nomenclature, Academic Press, Inc., 1992, pp. 55-60) such as e.g.:

malate oxidase 1.1.3.3, glucose oxidase (GOD) 1.1.3.4, hexose oxidase 1.1.3.5, cholesterol oxidase 1.1.3.6, aryl-alcohol oxidase 1.1.3.7, L-gluconolacton oxidase 1.1.3.8, galactose oxidase 1.1.3.9, pyranose oxidase 1.1.4.10, L-sorbose oxidase 1.1.3.11, alcohol oxidase 1.1.3.12, choline oxidase 1.1.3.17, secundary alcohol oxidase 1.1.3.18, glycerol-3-phosphate oxidase 1.1.3.21, xanthin oxidase 1.1.3.22, thiamin oxidase 1.1.3.23, L-galactonolacton oxidase 1.1.3.24, cellobiose oxidase 1.1.3.25, hydroxyphytanate oxidase 1.1.3.27, N-acetylhexosamine oxidase 1.1.3.29, polyvinyl-alcohol oxidase 1.1.3.30 and methanol oxidase 1.1.3.31. Particularly preferred among these enzymes are:

GOD, galactose Oxidase, alcohol oxidase und cellobiose oxidase.

As special (redox) enhancer compounds according to the invention the following compounds are preferably used like e.g.:

thiocyanates, isothiocyanates such as aryl-monoisothiocyanates, alkyl-monoisothiocyanates and the respective bis-compounds such as aryl-diisothiocyanates and alkyl-diisothiocyanates and isocyanates like alkyl- or aryl-monoisocyanates, and the respective bis-compounds like aryldiisocyanates, alkyldiisocyanates and furthermore e.g. such compounds as described in: Appendix 3, pp. 1637-1642 Lancaster (Clariant) research chemical catalogue 2004-2005.

Peroxidases together with peroxides, for example, can oxidize these compounds via thiocyanate to the strong oxidizing compounds hypothiocyanites and to the hypothiocyanic acids.

These new oxidoreductase/enhancer combinations (also laccase/O₂+enhancer) can be used for significant reductions of lignin content of pulps or generally for oxidation (redox) reactions.

It could also surprisingly be shown that beside these oxidations an activation of special coupling and/or cross-linking enhancer compounds according to the invention can be caused enzymatically in that way that coupling reactions of these compounds can take place generally on hydroxy-, thiol- or amino groups or especially hydroxyl groups e.g. in pulps (mainly on phenolic or aliphatic hydroxyl groups of the lignin part or hydroxyl groups of polysaccharide part).

This can lead either to a blocking of these groups (e.g. amination of phenols) or to an activation of these groups for simultaneous or successive coupling with suitable coupling reagents.

If bis-enhancer compounds (i.e. enhancer compounds with two reactive functionalities) are used cross-linking reactions can be initiated.

Furthermore coupling reactions and/or cross-linking reactions can be carried out using enzyme-based systems containing as enzyme component oxidoreductases but also special hydrolases e.g. lipases which can perform oxidizing reactions (as also described in the own patent application WO/9859108 and PCT/DE02/02035) and containing as coupling and/or cross-linking enhancer component

a) coupling and/or cross-linking precursors such as fatty acids and/or fats and/or fatty acid esters (if applicable) in combination with emulsifying agents (described in WO/9859108) generating bis-oxirane bearing fats, fatty acids.

b) reactive anchor substances like B-sulfooxyethylsulfone compounds ( sulphuric acid ester of the 2-Hydroxy-ethylsulfone) or generally sulfonyl-sulfamoyl or carbamoylalkylsulfoic acid group bearing compounds ( described e.g. in: in Zollinger: Color Chemistry, VCH, Weinheim, 1987) or

c) other coupling substances such as aldehydes, anhydrides , hydrazides, acryl derivatives vinyl derivatives, oxirane compounds, N-hydroxysuccinimidyl compounds etc. and their bis- and tris-compounds.

These compounds are able to react directly with relevant functionalities within the polymers according to the invention like amines, thiol, hydroxyl or carboxyl groups.

Other relevant coupling compounds according to the invention are described in: S. S. Wong ed.: Chemistry of Protein Conjugation and Cross-linking, CRC Press, 1991.

G. T. Hermanson et al. eds.: Immobilized Affinity Techniques, Academic Press, 1992 and:

G. T. Hermanson ed.: Bioconjugate Techniques, Academic Press, 1996.

Other preferred coupling and/or cross-linking enhancer compounds for the enzymatic coupling and/or cross-linking of special groups of the polymers according to the invention are:

I) coupling/ cross-linking of OH groups (e.g. polysaccharides):

a) reaction with epoxides and oxiranes or bi-functionally with bis-oxiranes

b) reaction with carbonyldiimidazoles (CDI)

c) reaction with N,N′-disuccinimidyl-carbonates (DSC)

d) reaction with isocyanates, diisocyanates or isothiocyanates oder diisothiocyanates

II) coupling/cross-linking of NH₂-groups (e.g. proteins):

a) reaction with isocyanates, diisocyanates or isothiocyanates oder diisothiocyanates

b) reaction with acyl azides

c) reaction with NHS ester compounds ( e.g.N-hydroxysuccinimide)

d) reaction with sulfonyl chlorides

e) reaction with aldehydes und glyoxales

f) reaction with carbonates

g) reaction with arylating reagents

h) reaction with imido ester compounds

i) reaction with carbodiimides

j) reaction with anhydrides

III) coupling/cross-linking of carboxyl groups (e.g. polysaccharides, proteins):

a) reaction with carbonyldiimidazoles (CDI)

b) reaction with carbodiimides

c) reaction with diazoalkanes and diacetyl compounds

IV) coupling/cross-linking of thiol groups( e.g. proteins):

a) reaction with haloacetyl and alkylhalide derivatives

b) reaction with maleimides

c) reaction with aziridines

d) reaction with acryloyl derivatives

e) reaction with arylating reagents

f) reaction with thiol/disulfide exchanging reagents

V) coupling/cross-linking of aldehydes- and ketones (e.g. polysaccharides and proteins etc.):

a) reaction with hydrazine derivatives

b) reaction with the aid of Schiff' scher base generation

c) reaction with the aid of reductive amination

d) reaction with the aid of Mannich condensation

VI) coupling/cross-linking of substrates with the aid of photo-reactive chemical reaction:

a) reaction with aryl azides und halogenated aryl azides

b) reaction with benzophenones

c) reaction with special diazo compounds

d) reaction with diazirine derivatives

Particularly preferred are homo-bifunctional cross-linkers bearing two equal coupling-relevant and groups or hetero-bifunctional cross-linkers bearing two different coupling-relevant end groups or tri-functional cross-linkers.

In all cases different functional substrate groups can be coupled with each other.

The main differences between the mentioned enzyme-controlled reactions (coupling/ cross-linking reactions) according to the invention in comparison to pure chemically-driven reactions are that one can work without further catalysts and that the velocity of the reactions is often much faster with significantly higher yields at significantly lower chemical charge and therefore lower costs.

In this respect particularly preferred enzyme-based coupling and/or cross-linking systems are modified HOS (hydrolase mediated oxidation system), described in WO/98/59108 (called there ECS).

It is possible by appropriate combination of hydrolases (mainly lipases), fatty acids or fats and H₂O₂ to generate per-fatty acids which can spontaneously form oxiranes to oligo-oxiranes where double bounds in unsaturated fatty acids/fats are available (see also in: U. Bornscheuer ed.: Enzymes in Lipid Modification, Wiley-VCH, 2000.

These enzymatically formed oxirane compounds (bearing single or multiple oxirane groups dependent on the number of available double bounds) can be used as coupling or cross-linking reagents for the reaction with hydroxyl, amine, or thiol groups within the coupling substrates.

Applications of the enzymatic oxidation systems (redox systems) and the enzyme-based coupling and/or cross-linking systems according to the invention

The mentioned oxidations systems (enzymes+(red/ox enhancer compounds) should be preferably used for bleaching and/or delignification of pulps for bleaching of textiles (cotton wool, wool), also for denim bleaching of jeans garments and for the bleaching in detergents.

A particularly preferred application is the use of the enzyme-based systems in combination with the coupling and /or cross-linking enhancer compounds according to the invention for coupling and/or cross-linking reactions with compounds which should be modified such as natural, (i.e. having natural origin) or artificial (i.e. synthetically produced) monomers to polymers or mixtures of natural and artificial polymers or fibre materials (preferably lignocellulose-containing, cellulose-containing or protein-like natural polymers) and with compounds which are coupled and/or cross-linked such as the same or similar compounds like natural, (i.e. having natural origin) or artificial (i.e. synthetically produced) monomers to polymers or mixtures of natural and artificial polymers or fibre materials (preferably lignocellulose-containing, cellulose-containing or protein-like natural polymers), especially with property-changing compounds such as monomer to polymer substances (natural or synthetic) either simultaneously or one after the other or

with compounds belonging to other substance groups like e.g. UV absorbing substances, radical scavengers.

The mentioned polymer compounds (i.e. compounds which should be modified and compounds which are coupled and/or cross-linked) according to the invention are preferably bio-polymers derived from plant, animal or microbial material as described e.g. in: Rauen, H. M., ed.: Biochemisches Taschenbuch, Springer Verlag, 1964; Elias, H-G. ed.:

Makromoleküle (Band 1 und 2), Hüthing & Wepf Verlag, 1992; Nuhn, P. ed.:

Naturstoffchemie, S. Hirzel Verlag, 1997 and Steinbüchel, A. ed.: Biopolymers, Vol. 1-10, Wiley-VCH, 2003.

These polymers according to the invention can be preferably complex, less complex including relatively uniform polysaccharides and/or preferably complex, less complex including relatively uniform polyamine compounds or proteins or protein-like substances and/or preferably complex, less complex including relatively uniform lignins, lignans and/or huminic substancen and/or preferably complex, less complex including relatively uniform polyester compounds such as poly-lactic acids or poly-glycolic acids, poly-ε-caprolactone compounds, poly-β-hydroxybutyric acid, poly-β-hydroxyvaleric acid, poly-dioxanones, poly-ethylenetherephthalates, poly-malonic acids , poly-tartaric acids, poly-(orthoester) compounds, poly-anhydrides, poly-cyanoacrylates, poly-(phosphoester) compounds and poly-phosphazenes and/or polyisoprenoides and/or fats or fatty acids and/or polynucleotides like dsesoxyribonucleinic acids or ribonucleinic acids, mixed polymers such as lipopolysaccharides, glycoproteines, glycolipides, lipoproteines or derivatives of the mentioned compounds.

Preferred polysaccharides according to the invention are particularly such as described in: Rauen, H. M.: Biochemisches Taschenbuch, pp. 718-734, 1964, such as:

starch and starch derivatives, amylopectin, glycogen, lichenan, pustulan, laminarin, lutean, yeast glucan, nigeran, pullulan, scleroglucan, curdlan, gellan, emulsan, acetan, welan, cellulose and cellulose derivatives inclusive pulps, dextrans and dextran derivatives, mannan, particularly yeast mannan, xylan, galaktan, araban, xanthan, tapioka, inulin and other fructosans of the inulin typ, levan, arabinogalactan, glucomannan, galactomannan, galactoglucomannan, phosphomannan, fucan, agar, agarose, cyclodexrin, carrageenan, pectin (not esterified and esterified), algin, chitin, chitosan, heparin, teichoic acid, hyaluronic acid chondroitin sulfate, carobin, and plant gums such as:

gum arabicum, gum tragacanth, gum karaya, gum ghatti, gum damar, gum locust bean, gum rosin, gum elemi, gum guaiac, gum guar, gum mastic, gum storax, gum pontianak etc., derivatives of the mentioned polysaccarides or mixtures.

Furthermore particularly preferred are proteins of animal, plant and microbial origin, also described in: Rauen, H. M.: Biochemisches Taschenbuch, pp. 778-813, 1964, such as animal proteins like albumin, plasmin, globulin, fibrinogen, thrombin, milk proteins like casein, lactalbumin, lactglobulin, animal proteins like collagen, keratin, fibroin, actin, myosin, elastine, gelatin, silk and wool, plant proteins such as grain proteins like hordein, glutenin etc., soy proteins, phaseolins, legumins etc. or poly-(α-aminoacids).

An other preferred application of the mentioned enzyme based systems according to the invention for coupling and/or cross-linking is the prevention or reduction of the yellowing of wood, pulp, textiles, plastics, paintings, carpet-floors or other materials caused by the influence of light (mainly UV), and/or oxygen and/or temperature.

Particularly preferred is the yellowing inhibition of pulps particularly high yield pulps, i.e, pulps with a high lignin content which is mainly responsible for the strong tendency to become yellow.

Such high yield pulps are e.g. unbleached TMP, CTMP or groundwood pulps or bleached pulps like BTMP, BCTMP or bleached groundwood

For the explanation of the effects see in: C. Heitner et al., eds.: Inhibition of Light Induced Yellowing of Lignin-Containing Paper, ACS Series, 1993.

The mentioned polymer substances can preferably coupled with UV-absorbing compounds, preferred with benzophenones and derivatives, benzotriazoles and derivatives, p-aminobenzoic acids and derivatives, cinnamic acids and derivatives, 2-phenylbenzimidazole compounds and derivatives and dibenzoylmethanes and derivatives.

Additionally preferred are radical scavengers such as compounds belonging to the group of hydroxylamines or NOH-compounds, particularly preferred such compounds belonging to the group of nitroxyl radicals like TEMPO compounds and/or nitrones or generally suitable antioxidants.

The mentioned polymer substances can also preferably coupled with optical brightener compounds such as derivatives of the flavonic acid, umbelliferon compounds cumarin compounds or such compounds described in: Jacoby et al. Detergents and Textile Washing; VCH, 1987.

For the application of the enzyme-based coupling and/or cross-linking also enzyme/enhancer systems should be generally used as described in the own patent applications: WO/98/59108, PCT/DE02/02035, PCT/DE03/00201 and DE 10215277.2.

In the following the invention is explained with the aid of examples but the invention should not restricted to these examples.

### EXAMPLE 1

### Enzymatic Delignification of Softwood O₂-Delignified (Sulphate Pulp)

5 g, absolutely dry basis, of wood pulp (softwood, O₂-delignified), pulp consistency 30% (about 17 g moist) was added to solutions prepared as follows:

A) To 20 mL of tap water were added 2-5 kg Na-Isothiocyanate per ton pulp with agitation. The pH was adjusted with sulfuric acid and/or sodium hydroxide solution so that, after addition of the wood pulp and the enzyme, the pH was 4.5.

B) To 5 mL of tap water 5 g peroxidase (HRP) (calculated as pure enzyme protein) and 1.5 kg H₂O₂ pro ton pulp was added under stirring.

Solutions A and B were combined and diluted to 33 mL. After addition of the wood pulp, the material was mixed in a dough mixer for 2 minutes. The material was then transferred to a reaction vessel preheated to 50° C. and was allowed to incubate 1-4 hours under atmospheric pressure.

The material was washed over a nylon screen (30 μm) and extracted for 1.5 hours at 70° C., 2% consistency and using 8% NaOH per gram of wood pulp. The material was again washed after which the kappa number was determined.

A kappa number reduction of 37% could be reached.

### EXAMPLE 2

### Enzymatic Coating of BCTMP High-Yield Pulp for Prevention of Yellowing Caused by UV-Light

5 g, absolutely dry basis, of wood pulp (hardwood BCTMP), pulp consistency 30% (about 17 g moist) was added to solutions prepared as follows:

A) To 20 mL of tap water were added 2 kg Na-Isothiocyanate per ton pulp with agitation. The pH was adjusted with sulfuric acid and/or sodium hydroxide solution so that, after addition of the wood pulp and the enzyme, the pH was ca. 5.0.

B) To 5 mL of tap water 5 g peroxidase (HRP) (calculated as pure enzyme protein), 1 kg Amino-TEMPO, 2.5 kg TINUVIN 1130 (benztriazole-compound) and 1.5 kg H₂O₂ per ton pulp was added under stirring.

Solutions A and B were combined and diluted to 33 mL. After addition of the wood pulp, the material was mixed in a dough mixer for 2 minutes. The material was then transferred to a reaction vessel preheated to 50° C. and was allowed to incubate 1 hour under atmospheric pressure.

The material was washed over a nylon screen (30 μm) and handsheets were made.

The irradiation of handsheets from treated BCTMP pulps was performed using an UV light source (SUNTEST UV equipment of Fa. Atlas, Germany) of ca. 300 to 420 nm for 24 hours and the change in brightness was measured against an untreated sample for reference. A reduction of the yellowing of more than 15 ISO brightness % could be obtained.

1) Enzyme-based methods for carrying out oxidizing reactions (redox reactions) and
for carrying out coupling and/ or cross-linking reactions, characterized in, that
a) these oxidation, coupling and/or cross-linking reactions are carrying out using hydrolases such as lipases, esterases, proteases, amidases, transferases, acylases, glycosidases, glycotransferases or using oxidoreductases, such as preferably peroxidases, chloroperoxidases and laccases, either individually or in combination with one another; and
characterized in, that
b) the oxidizing reactions are performed using the mentioned enzymes together with
special (redox) enhancer compounds and characterized in, that
c) these coupling and /or cross-linking reactions are carrying out with compounds which should be modified such as natural (i.e. having natural origin) or artificial (i.e. synthetically produced) monomers to polymers or mixtures of natural and artificial polymers or fibre materials (preferably lignocellulose-containing, cellulose-containing or protein-like natural polymers)
in combination with compounds which are coupled and/or cross-linked such as the same or similar compounds like natural, (i.e. having natural origin) or artificial (i.e. synthetically produced) monomers to polymers or mixtures of natural and artificial polymers or fibre materials (preferably lignocellulose-containing, cellulose-containing or protein-like natural polymers) especially
in combination with property-changing compounds such as monomer to polymer substances (natural or synthetic) either simultaneously or one after the other or
with compounds belonging to other substance groups like e.g. UV absorbing substances, radical scavengers, etc., and characterized in, that
d) special coupling and/or cross-linking enhancer compounds or coupling and/or cross-linking precursor compounds which are the coupling and/or cross-linking agents activated by the enzymes are used.

2. Methods according to claim 1, characterized in, that as enzymes such of the group of 2.2, 2.4, 3.1, 3.2, 3.4, 3.5 (hydrolases) and/or oxidoreductases of the class 1 and particularly of the groups 1.10.3.2 (laccases), 1.11.1.7. (peroxidases) and 1.11.1.10 (chloroperoxidases) are used.

3. Methods according to claim 1, characterized in, that as redox enhancer compounds such substances from the groups of isocyanates, isothiocyanates, thiocyanates and of their bis-compounds are used.

4. Methods according to claim 1 and 3, characterized in, that as oxidation systems peroxidase/H₂O₂-systems, laccase/O₂-systems or hydrolase/H₂O₂/fat- or fatty acid systems (with or without ketone additives) are used together with isocyanates, isothiocyanates and/or thiocyanates and their bis-compounds as redox enhancer compounds and/or coupling and/or cross-linking enhancer compounds.

5. Methods according to claim 1, characterized in, that in combination with oxidoreductase or oxidative hydrolase-systems coupling and/or cross-linking enhancer compounds or coupling and/or cross-linking precursors such as fats, fatty acids, fatty acid esters (if applicable together with special emulgators),
such as reactive anchor substances like β-Sulfooxyethylsulfone compounds etc.,
such as coupling/cross-linking compounds like aldehydes, anhydrides, hydrazides, acryl derivatives, vinyl derivatives and
such as coupling/cross-linking compounds like oxirane compounds and
N-hydroxysuccimidyl-compounds and their bis- and tris-compounds are used.

6. Methods according to claim 1, characterized in, that in combination with oxidoreductase or oxidative hydrolase-systems coupling and/or cross-linking enhancer compounds or coupling and/or cross-linking precursors such as unsaturated fats, fatty acids and fatty acid ester compounds are used which can be converted by the reaction with suitable hydrolases (lipases) and H₂O₂ to per-fatty acids generating spontaneously the corresponding oxirane or bis-oxirane to oligo-oxirane compounds.

7. Methods according to claim 1, characterized in, that as applications bleaching /delignification of pulps, bleaching of textiles (cotton, wool), bleaching in detergents, coupling and/or cross-linking reactions on natural (i.e. having natural origin) or of artificial (i.e. synthetically produced polymers) monomers to polymers or of mixtures of natural and artificial polymers or of fibre materials of lignocellulose-containing, cellulose-containing or protein-like natural polymers or of fibre materials such as pulp, textiles like cotton and wool are carried out in combination with compounds which are coupled and/or cross-linked such as the same or similar compounds like natural, (i.e. having natural origin) or artificial (i.e. synthetically produced) monomers to polymers or mixtures of natural and artificial polymers or fibre materials (preferably lignocellulose-containing, cellulose-containing or protein-like natural polymers), especially bio-polymers derived from plant, animal or microbial material like polysaccharides and/or polyamine compounds and/or proteins or protein-like substances and/or lignins, lignans and/or huminic substancen and/or polyester compounds
and/or in combination with compounds which are coupled and/or cross-linked, such as compounds belonging to other substance groups like UV absorbing substances, radical scavengers.

8. Methods according to claim 1, characterized in, that the mentioned methods can be used for the protection or minimizing of the yellowing of wood pulps, particularly preferred high yield pulps, plastics, textiles, paintings or carpet floors and/or all material exposured to light (UV) etc. and/or oxygen, and/or temperature.

9. Methods according to claim 1, characterized in, that as coupling, cross-linking substances UV-absorbing compounds, preferably benzophenone and benzotriazole derivatives, and/or radical scavengers, preferred belonging to the group of hydroxylamines and/or NOH-compounds or particularly preferred belonging to the group of nitroxyl-radicals and/or nitrones and/or optical brightener like derivatives of flavones, umbelliferones other cumarin compounds are used.

10. Methods according to claim 1, characterized in, that the applications are performed at pH 2-10 (preferably at pH 3-8), at 10 to 100° C. (preferably 20-70° C.) for 10 minutes to 36 hours (preferably 0.5 to 8 hours) and at consistencies of 0.5% to 40% (preferably 1% to 12.5%) in a water-containing environment or in mixed systems: water/solvents under air, oxygen or other gases like CO₂ etc. under pressure or atmospheric pressure.
